(19) United States
(12) Patent Application Publication
(10) Pub. No.: US 2018/0258115 A1
(43) Pub. Date: Sep. 13, 2018

(54) BRØNSTED-ACIDIC FLUOROALKYL PHOSPHONATES

(71) Applicant: Merck Patent GmbH, Darmstadt (DE)

(72) Inventors: Nikolai (Mykola) IGNATIEV, Duisburg (DE); Peter BARTHEN, Rheinberg (DE); Bernd BREDENHAGEN, Frankfurt am Main (DE); Michaela Katharina MEYER, Krefeld (DE); Walter FRANK, Wuppertal (DE)

(73) Assignee: Merck Patent GmbH, Darmstadt (DE)

(21) Appl. No.: 15/759,987

(22) PCT Filed: Aug. 30, 2016
(86) PCT No.: PCT/EP2016/001463

§ 371 Date: Mar. 14, 2018

(30) Foreign Application Priority Data
Sep. 15, 2015 (EP) 15185154.0

(51) Int. Cl. C07F 9/48; C07C 45/61; C07C 67/00; C07C 45/46; C07C 67/03; B01J 31/02

(52) U.S. Cl. CPC C07F 9/4816; C07C 45/61; C07C 67/00; C07C 45/46; B01J 2231/32; B01J 31/0261; C07C 2601/14; B01J 2231/4205; C07C 67/03

(57) ABSTRACT

The invention relates to Brønsted-acidic fluoroalkyl phosphonates as bifunctional catalysts and to processes for the preparation thereof.

The invention relates to Brønsted-acidic fluoroalkyl phosphonates as bifunctional catalysts, and to processes for the preparation thereof.

Catalysis using Lewis acids is a widespread method in organic synthesis and of outstanding importance for the industrial preparation of various substances. The numerous important industrial processes that are catalysed by Lewis acids include, for example, Friedel-Crafts alkylations and acylations of aromatic compounds, Gattermann-Koch reactions, Beckmann and Fries rearrangements, Mukaiyama aldol condensations [Acid Catalysis in Modern Organic Synthesis, H. Yamamoto and K. Ishihara (Eds.), WILEY-VCH, Weinheim, 2008].

G. N. Lewis defines an acid as a substance which is able to act as electron-pair acceptor. In accordance with this definition, Lewis acids are electron-deficient molecules or ions. The Lewis-acidic catalysts usually used, such as AlCl₃, TiCl₄, ZnCl₂ and BF₃ diethyl etherate, are moisture-sensitive and generally cannot be recovered after completion of the reaction.

Catalysis with Brønsted acid catalysts is a further useful concept in organic synthesis [for examples see: G. A. Olah, G. K. S. Prakash, A. Molnar, J. Sommer, Superacid Chemistry, Second Edition, WILEY, 2009; M. Beller, A. Renken, R. A. van Santen (Eds), Catalysis. From Principles to Applications, WILEY-VCH, 2012]. Brønsted acids are assigned to different classes depending on their strength. Besides weak acids, such as, for example, acetic acid, and strong acids, such as trifluoroacetic acid, hydrochloric acid or sulfuric acid, there is the class of superacids, which have a higher acid strength than sulfuric acid, which includes, for example, trifluoromethane-sulfonic acid, CF₃SO₃H. There are numerous reactions which are catalysed by Brønsted acids, for example Friedel-Crafts alkylation and acylation, the Diels-Alder reaction, the aldol condensation, the hydration of alkenes, cyclisation and polymerisation processes. In many cases, the reaction can be catalysed by relatively weak Brønsted acids, but in some cases the use of superacids, such as, for example, perfluoroalkanesulfonic acids, is necessary in order that the reaction proceeds completely in a satisfactorily short time [for some examples see: G. A. Olah, G. K. S. Prakash, A. Molnar, J. Sommer, Superacid Chemistry, Second Edition, WILEY, 2009]. These frequently used acids are very stable compounds and, in particular, the substances having long perfluoroalkyl groups are persistent. Reservations owing to their poor biodegradability therefore limit the practical usability of perfluoroalkanesulfonic acids having long “perfluorinated chains”.

A modern concept for the simplification of chemical processes is based on the use of “one-pot” or “cascade” (or “domino”) reactions [L. F. Tietze (Ed.), Domino Reactions. Concepts for Efficient Organic Synthesis, WILEY-VCH, 2014; V. A. Smit, A. D. Dilmann, Fundamentals of Modern Organic Synthesis, BINOM. Moscow, 2009; A. Hassner, I. Namboothiri, Organic Syntheses Based on Name Reactions, Third Edition, Elsevier Ltd., 2012; J. J. Li, Name Reactions, Springer-Verlag, Berlin, Heidelberg, 2003]. The concept allows the production time to be shortened and the energy consumption to be reduced, in particular in the case of multistep syntheses of fine chemicals.

In many cases, both Lewis acid and Brønsted acid catalysis is necessary in order to accelerate different individual steps in these multistep reactions. Substances which have two different catalytically active regions and are capable of catalysing two different types of reaction are usually called bifunctional catalysts. A known example of a catalyst of this type is arylboronic acid, ArB(OH)₂. In order to increase the Lewis acidity of the central boron atom, the aryl group should preferably have electron-withdrawing substituents. Arylboronic acids are not strong Brønsted acids, but can nevertheless be employed successfully for catalysis in some cases, in order, for example, to esterify α-hydroxycarboxylic acids [T. Maki, K. Ishihara, H. Yamamoto, Tetrahedron 2007, 63, 8645-8657] or in order to prepare the corresponding amides from phenylacetic acid and amines [R. M. Al-Zoubi, O. Marion, D. G. Hall, Angew. Chem. Int. Ed. 2008, 47, 2876-2879]. The catalytic activity of neutral arylboronic acids is greatly reduced in polar solvents. This restricts the choice of soluble substrates [Y. Morita, Combining transition metal catalysis and organocatalysis, 2011]. The combination of BINOL-phosphoric acids with their salts is a further approach for the preparation of bifunctional catalysts [D. Parmar, E. Sugiono, S. Raja, M. Rueping, Chem. Rev. 2014, 114, 9047-9153]:

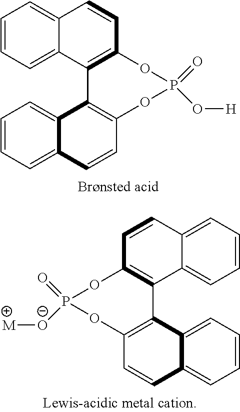

Unsubstituted BINOL-phosphoric acid is not a strong acid, with a pKₐ in acetonitrile of 13.3, compared with picric acid, with a pKₐ in acetonitrile of 11.0. It is furthermore described that the acid strength can be increased by substitution at the 3,3′-positions. This catalytic system catalyses various organic reactions, for example additions onto imines, carbonyl groups and activated alkanes, intramolecular cyclisations, hetero-Diels-Alder reactions.

In order to provide both Brønsted acid and Lewis acid catalysis, the corresponding BINOL-phosphoric acid must be employed together with a metal salt. This makes both product isolation and also catalyst regeneration more difficult. The combination of Brønsted acidic and Lewis acidic function in a single compound would be advantageous.

Only very few compounds containing Brønsted acid and Lewis acid groups are known, for example iron hydrogensulfate Fe(HSO₄)₃, aluminium hydrogensulfate Al(HSO₄)₃ or zirconium hydrogenphosphate Zr(HPO₄)₂.

These inorganic bifunctional catalysts have been tested in Friedel-Crafts acylations [P. Salehi, M. M. Khodaei, M. A. Zolfigol, S. Sirouszadeh Synthetic Communications 2003, 33, 8, 1367-1373; P. Salehi, M. M. Khodaei, M. A. Zolfigol, S. Sirouszadeh, Bull. Chem. Soc. Jpn. 2003, 76, 1863-1864], in the synthesis of 14-aryl- or alkyl-14H-dibenzo[a,j]xanthenes [H. Eshghi, M. Bakavoli, H. Moradi, Chinese Chemical Letters 2008, 19, 1423-1426], and the trimethylsilylation of alcohols and phenols [F. Shirini, M. A. Zolfigol, A.-R. Abri, Monatsh. Chemie 2008, 139, 17-20]. The disadvantages of these inorganic bifunctional catalysts are the poor solubility in organic solvents and the low catalytic activity. It is usually necessary to employ 15-25 mol % of catalyst. Hydrogensulfate and hydrogenphosphate are coordinating anions and therefore reduce the Lewis-acidic catalytic activity of the central cation, for example Fe³⁺ or Al³⁺. In addition, the Brønsted acidity of the HSO₄⁻ ion is considerably limited. The pKₐ value for the second ionisation step of H₂SO₄ is 1.92.

There therefore also continues to be a demand for economically attractive bifunctional catalysts, as described above.

The object of the present invention is therefore to develop alternative bifunctional catalysts which preferably have advantages over bifunctional catalysts known to date, for example a lower use concentration in the reactions to be catalysed.

Surprisingly, it has been found that fluoroalkyl hydrogenphosphonates are suitable bifunctional catalysts.

The fluorinated alkyl groups, preferably perfluorinated alkyl groups, on the phosphorus increase the Brønsted acidity of these compounds, and the weak coordination of the metal cation supports a high Lewis acidity of these compounds, so that they provide extraordinarily high bifunctional catalytic activity.

The invention therefore relates firstly to compounds of the formula I

[Kt]^{z+}z[R_{f}P(O)(OH)O]⁻ I,

where
R_{f} corresponds to the formula CₙF₍₂ₙ₊₁₎₋₍ₘ₊ₖ₎HₘXₖ,
X denotes Cl, Br or I,
n denotes 1, 2, 3, 4, 5, 6, 7, 8, 9, 10, 11 or 12,
m denotes 0, 1, 2, 3 or 4,
k denotes 0, 1 or 2,
with the proviso that, if n denotes 1, m denotes 0, 1 or 2 and/or k denotes 0, 1 or 2 and
[Kt]^{z+} denotes a singly, doubly, triply or multiply positively charged metal atom.

The invention furthermore relates to the use of the compounds of the formula I

[Kt]^{z+}z[R_{f}P(O)(OH)O]⁻ I,

where
R_{f} corresponds to the formula CₙF₍₂ₙ₊₁₎₋₍ₘ₊ₖ₎HₘXₖ,
X denotes Cl, Br or I,
n denotes 1, 2, 3, 4, 5, 6, 7, 8, 9, 10, 11 or 12,
m denotes 0, 1, 2, 3 or 4,
k denotes 0, 1 or 2,
with the proviso that, if n denotes 1, m denotes 0, 1 or 2 and/or k denotes 0, 1 or 2 and
[Kt]^{z+} denotes a singly, doubly, triply or multiply positively charged metal atom,
as bifunctional catalysts.

The invention furthermore relates to the bifunctional catalysts of the formula I

[Kt]^{z+}z[R_{f}P(O)(OH)O]⁻ I,

where
R_{f} corresponds to the formula CₙF₍₂ₙ₊₁₎₋₍ₘ₊ₖ₎HₘXₖ,
X denotes Cl, Br or I,
n denotes 1, 2, 3, 4, 5, 6, 7, 8, 9, 10, 11 or 12,
m denotes 0, 1, 2, 3 or 4,
k denotes 0, 1 or 2,
with the proviso that, if n denotes 1, m denotes 0, 1 or 2 and/or k denotes 0, 1 or 2 and
[Kt]^{z+} denotes a singly, doubly, triply or multiply positively charged metal atom, for use in organic synthesis.

Suitable groups R_{f} in the compounds of the formula I are accordingly linear or branched fluoroalkyl groups having 1 to 12 C atoms, which may be substituted by Cl, Br or I.

A linear or branched alkyl group having 1 to 12 C atoms is, for example, methyl, ethyl, isopropyl, propyl, butyl, sec-butyl or tert-butyl, pentyl, 1-, 2- or 3-methylbutyl, 1,1-, 1,2- or 2,2-dimethylpropyl, 1-ethylpropyl, n-hexyl, n-heptyl, n-octyl, n-nonyl, n-decyl, n-undecyl or n-dodecyl.

A linear or branched fluoroalkyl group is a linear or branched fluorinated alkyl group having 1 to 12 C atoms in which at least one H atom of a linear or branched alkyl group having 1 to 12 C atoms is replaced by an F atom. It is also possible for all H atoms to be replaced by F atoms. Alkyl groups of this type are accordingly linear or branched perfluoroalkyl groups having 1 to 12 C atoms.

In accordance with the invention, the linear or branched fluoroalkyl group having 1 to 12 C atoms may be substituted by Cl, Br or I atoms, where the number of these substituents where they occur denotes 1 or 2.

R_{f} is preferably trifluoromethyl, difluoromethyl, fluoromethyl, difluorochloromethyl, difluorobromomethyl, monochloromonofluoromethyl, 2,2,2-trifluoroethyl, 2,2-difluoroethyl, 2-chloro-2-fluoroethyl, 1-chloro-2,2-difluoroethyl, pentafluoroethyl, heptafluoroisopropyl, n-heptafluoropropyl, n-nonafluorobutyl and all other perfluoroalkyl groups having 5 to 12 C atoms.

In a preferred embodiment of the invention, R_{f} is a linear or branched perfluoroalkyl group having 1 to 12 C atoms.

The invention furthermore relates to a compound or a bifunctional catalyst of the formula I in which R_{f} denotes a linear or branched perfluoroalkyl group having 1 to 12 C atoms.

In a particularly preferred embodiment of the invention, R_{f} is a linear or branched perfluoroalkyl group having 1 to 4 C atoms.

In a very particularly preferred embodiment of the invention, R_{f} is pentafluoroethyl or n-nonafluorobutyl.

In a preferred embodiment of the invention, the cation [Kt]^{z+} of the compounds of the formula I is selected from cations of the metals Li, Na, Ca, Mg, Ag, Fe, Co, Ni, Cu, Au, Al, In, Sn, Zn, Bi, Rh, Ru, Ir, Pd, Pt, Os, Cr, Mo, W, V, Nb, Ta, Ti, Zr, Hf, Y, Yb, La, Sc, Lu, Ce, Nd, Tb, Er, Eu or Sm.

In accordance with the invention, the cation [Kt]^{z+} may also include cations of the metals, as described above, which are coordinated to further ligands, such as, for example, to Cl, OH, CO, cyclopentadienyl, phosphine, heterocyclic carbenes or alkenyl ligands, which stabilise the desired oxidation state of the metal.

The invention therefore furthermore relates to the compounds or bifunctional catalysts of the formula I, as described above or described as preferred, in which [Kt]^{z+} is selected from cations of the metals Li, Na, Ca, Mg, Ag, Fe, Co, Ni, Cu, Au, Al, In, Sn, Zn, Bi, Rh, Ru, Ir, Pd, Pt, Os, Cr, Mo, W, V, Nb, Ta, Ti, Zr, Hf, Y, Yb, La, Sc, Lu, Ce, Nd, Tb, Er, Eu or Sm.

Particularly preferred compounds of the formula I are compounds in which the cation [Kt]^{z+} is selected from cations of the metals Li, Ca, Mg, Fe, Ni, Co, Cu, Au, Al, In, Zn, Bi, Rh, Ru, Ir, Pd, Pt or Y, Sc, Yb, Tb, Lu, Sm.

Very particularly preferred compounds of the formula I are compounds in which the cation [Kt]^{z+} is selected from cations of the metals Fe and Al.

The invention furthermore relates to a process for the preparation of compounds of the formula I, as described above or preferably described, characterised in that a compound of the formula II

R_{f}P(O)(OH)₂ II,

where
R_{f} corresponds to the formula CₙF₍₂ₙ₊₁₎₋₍ₘ₊ₖ₎HₘXₖ,
X denotes Cl, Br or I,
n denotes 1, 2, 3, 4, 5, 6, 7, 8, 9, 10, 11 or 12,
m denotes 0, 1, 2, 3 or 4,
k denotes 0, 1 or 2,
with the proviso that, if n denotes 1, m denotes 0, 1 or 2 and/or k denotes 0, 1 or 2, or has a meaning indicated as preferred, as described above, is reacted with a metal which forms the future metal cation [Kt]^{z+}, or a compound of the formula III

[Kt]^{z+}z/b[An]^{b-} III,

where [Kt]^{z+} denotes a singly, doubly, triply or multiply positively charged metal atom and has a meaning preferably indicated above, and
[An]^{b-} denotes [Cl]⁻, [OH]⁻ [CO₃]²⁻ or [O]²⁻,
where equimolar amounts of compounds of the formula II and of the formula III or of the metal are employed, which is determined by the valence of the cation [Kt]^{z+} in the compound of the formula I. The variable b corresponds to the valence of the anion.

The anion [An]^{b-} of the compound of the formula III is preferably [Cl]⁻.

Compounds of the formula III are commercially available.

The metals to be used in accordance with the invention are commercially available.

The metals can be employed in any existing form of metal particles, for example in the form of powders or turnings. The use of powders is preferred in accordance with the invention.

Compounds of the formula II are commercially available or can be prepared by processes which are known to the person skilled in the art, for example in accordance with N. V. Ignat'ev et al, Chimica Oggi/Chemistry Today 2011, 29, 5, 42-44.

The reaction of the compounds of the formula II with compounds of the formula III or metals can be carried out in the presence of water or an organic solvent or without the presence of water or an organic solvent.

The invention therefore furthermore relates to the process as described above, characterised in that the reaction is carried out in the presence or without the presence of water or an organic solvent.

Suitable solvents are, for example, ethers, such as diethyl ether, methyl tert-butyl ether or 1,4-dioxane, or ketones, such as, for example, acetone, or amides, such as, for example, dimethylformamide or dimethylacetamide.

In the case of the reaction with compounds of the formula III, as described above, which are sensitive to hydrolysis, for example AlCl₃, it is preferred if the reaction, as described above or described below, takes place in an inert-gas atmosphere.

The water content of the reagents and of the inert-gas atmosphere in this embodiment is a maximum of 1000 ppm. It is particularly preferred if the water content of the reagents and of the atmosphere is less than 500 ppm, very particularly preferably a maximum of 100 ppm.

In the case of the reaction of compounds of the formula II with a metal, in particular with an alkali metal or alkaline-earth metal, as described above, it is preferred if the oxygen content and the water content are a maximum of 1000 ppm. It is particularly preferred if the oxygen content is less than 500 ppm.

The conditions with respect to the water content and the oxygen content do not apply to the reaction in water and do not apply to the work-up after successful reaction of compounds of the formula II, as described below.

In general, the starting materials are mixed at low temperature of 0° C. to 10° C. using diethyl ether or at room temperature (20° C. to 25° C.). Depending on the metal employed or the compound of the formula III employed, as described above, the hydrogen, hydrogen chloride or CO₂ formed is preferably removed from the system, for example by means of a stream of inert gas or by co-distillation with a suitable solvent which complexes, for example, the hydrogen chloride formed. A preferred inert gas is dry air, dry nitrogen or argon. Where appropriate, after removal of the hydrogen chloride, the product is heated to a temperature between 50° C. and 200° C. and dried.

A suitable solvent for the complexing of hydrogen chloride is, for example, the organic solvent which has already been selected for the reaction, or a mixture thereof.

The distillation is then carried out correspondingly at temperatures which is necessary for the solvent/hydrogen chloride mixture. The co-distillation operation is preferably continued until chloride ions can no longer be detected in the distillate.

After removal of the hydrogen chloride, the mixture is optionally heated to a temperature between 50° C. and 200° C. and the product is dried. Alternatively, the product formed can be dried in vacuo at room temperature or temperatures up to 50° C.

If a solvent is selected from which the product of the formula I formed precipitates out, the product is filtered off and subsequently dried at a higher temperature than the boiling point of the solvent selected.

If appropriate, this is preferably followed by a suitable purification method, for example a recrystallisation from a suitable solvent or a sublimation.

In an alternative preparation method, a compound of the formula I, as described above, can be prepared by a disproportionation reaction. Corresponding fluoroalkyl phosphonates of the formula IV, as described below, can accordingly be reacted with a corresponding amount of the acid of the formula II. The disproportionation reaction can be carried out in water or in an organic solvent or without the presence of water or the organic solvent.

Suitable temperatures are 10° C. to 150° C., preferably room temperature to 100° C.

The invention therefore furthermore relates to the process for the preparation of the compounds of the formula I, as described above or preferably described, characterised in that a compound of the formula

### IV

[Kt]^{z+}z/2[R_{f}P(O)O₂]²⁻ IV,

is reacted in a disproportionation reaction with a compound of the formula II

R_{f}P(O)(OH)₂ II,

where
R_{f} corresponds to the formula CₙF₍₂ₙ₊₁₎₋₍ₘ₊ₖ₎HₘXₖ,
X denotes Cl, Br or I,
n denotes 1, 2, 3, 4, 5, 6, 7, 8, 9, 10, 11 or 12,
m denotes 0, 1, 2, 3 or 4,
k denotes 0, 1 or 2,
with the proviso that, if n denotes 1, m denotes 0, 1 or 2 and/or k denotes 0, 1 or 2, or has a meaning indicated as preferred, as described above, and where [Kt]^{z+} denotes a singly, doubly, triply or multiply positively charged metal atom or has a meaning preferably indicated above.

Suitable solvents are, for example, ethers, such as methyl tert-butyl ether or 1,4-dioxane, or amides, such as, for example, dimethylformamide or dimethylacetamide.

If a solvent is selected from which the product of the formula I formed precipitates out, the product is filtered off and subsequently dried at a higher temperature than the boiling point of the solvent selected

The disproportionation reaction is, where appropriate, preferably followed by a suitable purification method, for example an extraction, or a recrystallisation from a suitable solvent or a sublimation and subsequent drying.

However, the dried products of the formula I can also be employed without further purification in the reactions to be catalysed.

Accordingly, the invention furthermore relates to the bifunctional catalysts of the formula I, as described above or preferably described, for use in organic synthesis.

In a preferred embodiment of the invention, the reactions to be catalysed are selected from a condensation reaction, alcoholysis, aldol reaction, Mukaiyama aldol reaction, Gattermann-Koch reaction, Beckmann and Fries rearrangement, Friedel-Crafts acylation, Friedel-Crafts alkylation, Mannich reaction, Diels-Alder reaction, aza-Diels-Alder reaction, Baylis-Hillman reaction, Reformatsky reaction, Claisen rearrangement, Prins cyclisation reaction, allylation of carbonyl compounds, cyanation of aldehydes and ketones, cyanosilylation of aldehydes and ketones, 1,3-dipolar cycloaddition, hydration of alkenes, cyclisation reaction, polymerisation, Michael reaction, oxidation and reduction reactions.

The invention therefore furthermore relates to the use of compounds of the formula I in reactions selected from a condensation reaction, alcoholysis, aldol reaction, Mukaiyama aldol reaction, Gattermann-Koch reaction, Beckmann and Fries rearrangement, Friedel-Crafts acylation, Friedel-Crafts alkylation, Mannich reaction, Diels-Alder reaction, aza-Diels-Alder reaction, Baylis-Hillman reaction, Reformatsky reaction, Claisen rearrangement, Prins cyclisation reaction, allylation of carbonyl compounds, cyanation of aldehydes and ketones, cyanosilylation of aldehydes and ketones, 1,3-dipolar cycloaddition, hydration of alkenes, cyclisation reaction, polymerisation, Michael reaction, oxidation and reduction reactions.

The invention therefore furthermore relates to the bifunctional catalysts of the formula I for use in Lewis acid-catalysed and/or and/or Brønsted acid-catalysed reactions or domino reactions selected from a condensation reaction, alcoholysis, aldol reaction, Mukaiyama aldol reaction, Gattermann-Koch reaction, Beckmann and Fries rearrangement, Friedel-Crafts acylation, Friedel-Crafts alkylation, Mannich reaction, Diels-Alder reaction, aza-Diels-Alder reaction, Baylis-Hillman reaction, Reformatsky reaction, Claisen rearrangement, Prins cyclisation reaction, allylation of carbonyl compounds, cyanation of aldehydes and ketones, cyanosilylation of aldehydes and ketones, 1,3-dipolar cycloaddition, hydration of alkenes, cyclisation reaction, polymerisation, Michael reaction, oxidation and reduction reactions.

The compounds of the formula I are preferably employed in a sub-stoichiometric amount of catalyst of 0.001 to 20 mol %, based on the starting material, and depending on the type of metal cation [Kt]^{z+}. The compounds of the formula I, as described above or described as preferred, are particularly preferably employed in an amount of 0.1 to 10 mol %, particularly preferably in an amount of 1, 2, 5 or 10 mol %. The person skilled in the art in the area of catalysis is able to select the optimum amount of catalyst, depending on the type of metal cation [Kt]^{z+}, for the corresponding reaction to be catalysed and the other reaction conditions optimally.

Depending on the reaction to be catalysed, a suitable solvent should be selected which preferably takes into account the specific solution properties of the compounds of formula I, as described above or described as preferred.

Suitable protic solvents on use of the bifunctional catalysts according to the invention are water, ethanol, methanol, isopropanol, ethylene glycol or polyethylene glycol.

Suitable aprotic solvents on use of the bifunctional catalysts according to the invention are acetonitrile, diethyl ether, methyl tert-butyl ether, tetrahydrofuran, 2-methyltetrahydrofuran, monoglyme, diglyme, triglyme, dimethylformamide, dimethylacetamide, dimethyl sulfoxide, N-methylpyrrolidone, dichloromethane, 1,2-dichlormethane, benzene, toluene, hexane, heptane, petroleum ether or mixtures of the said solvents.

The class of the ionic liquids are also suitable as solvents on use of the bifunctional catalysts according to the invention.

An ionic liquid is taken to mean salts which generally consist of an organic cation and an inorganic anion. They do not contain any neutral molecules and usually have melting points below 373 K [Wasserscheid P, Keim W, Angew. Chem. 112, 2000, 3926]. Due to their salt character, ionic liquids have unique substance properties, such as, for example, a low vapour pressure, a liquid state over a broad temperature range, are non-flammable, exhibit high electrical conductivity and high electrochemical and thermal stability.

Suitable ionic liquids as solvents on use of the Lewis acid catalysts according to the invention are ionic liquids which have an organic cation and whose anion is selected from the group [R₁SO₃]⁻, [R₂COO]⁻, [R₂SO₃], [R₁OSO₃]⁻, [BF₄]⁻, [HSO₄]¹⁻, [(R₁)₂P(O)O]⁻, [(R₂)₂P(O)O]⁻, [R₂P(O)O₂]²⁻, [(FSO₂)₂N]⁻, [(R₂SO₂)₂N]⁻, [(R₂SO₂)₃C]⁻, [(FSO₂)₃C]⁻, [P(R₂)_{y}F_{6-y}]⁻, [BFₓ(R₂)₄₋ₓ]⁻, [BFₓ(CN)₄₋ₓ]⁻, [B(R₁)ₐ(CN)₄₋ₐ]⁻, [B(R₂)F₂(CN)]⁻ or [B(R₂)F(CN)₂]⁻,

where R₁ in each case, independently of one another, denotes a linear or branched alkyl group having 1 to 12 C atoms,
R₂ in each case, independently of one another, denotes a partially fluorinated or perfluorinated linear or branched alkyl group having 1 to 12 C atoms or pentafluorophenyl,
x denotes the integer 0, 1, 2 or 3,
y denotes the integer 0, 1, 2, 3 or 4 and
a denotes the integer 1 or 2.

A perfluorinated linear or branched alkyl group having 1 to 4 C atoms is, for example, trifluoromethyl, pentafluoroethyl, n-heptafluoropropyl, iso-heptafluoropropyl, n-nonafluorobutyl, sec-nonafluorobutyl or tert-nonafluorobutyl. R₂ analogously defines a linear or branched perfluorinated alkyl group having 1 to 12 C atoms, including the above-mentioned perfluoroalkyl groups and, for example, perfluorinated n-hexyl, perfluorinated n-heptyl, perfluorinated n-octyl, perfluorinated ethylhexyl, perfluorinated n-nonyl, perfluorinated n-decyl, perfluorinated n-undecyl or perfluorinated n-dodecyl.

R₂ is preferably trifluoromethyl, pentafluoroethyl or nonafluorobutyl, very particularly preferably trifluoromethyl or pentafluoroethyl.

The variable y is preferably 1, 2 or 3, particularly preferably 3.

Preferred solvents are ionic liquids with the anions [P(R₂)_{y}F_{6-y}]⁻ and [R₂SO₃]⁻, where R₂ and y have a meaning indicated above or indicated as preferred.

Particularly preferred solvents are ionic liquids with the anions [P(C₂F₅)₃F₃]⁻ and [CF₃SO₃]⁻.

The organic cations are generally unrestricted and are preferably selected from imidazolium cations, pyridinium cations or pyrrolidinium cations, which may be appropriately substituted, as known from the prior art.

The suitability of the compounds of the formula I as bifunctional catalysts has been confirmed with reference to reaction time of the Michael reaction, a Friedel-Crafts acylation and the methanolysis of octyl acetate. These reaction types are representative of reactions catalysed by Lewis acids.

Even without further comments, it is assumed that a person skilled in the art will be able to utilise the following descriptions in the broadest scope. The preferred embodiments and examples should therefore merely be regarded as as descriptive disclosure which is absolutely not limiting in any way.

### Analysis:

NMR samples are measured either in a 5 mm (Ø_{A}) glass NMR tube or in a 3.7 mm (Ø_{A}) FEP inliner at 25° C. In the case of measurements in FEP the inliner is introduced into a 5 mm (Ø_{A}) precision thin-glass NMR tube (Wilmad 537). The lock substance, CD₃CN, is located in the glass NMR tube, i.e. between glass and FEP inliner, and is labelled with film measurement or solvent film below. The measurements are carried out in a 400 MHz Bruker Avance III spectrometer with a 9.3980 T cryomagnet and a 5 mm BBFO sample head. ¹H NMR spectra are measured in the ¹H/¹⁹F channel at 400.17 MHz. ¹⁹F and ³¹P NMR spectra are measured in the broad-band channel at 376.54 and 161.99 MHz. The ¹H NMR chemical shifts are relative to tetramethylsilane (TMS) and arise for the solvents D₂O (4.81 ppm), CDCl₃ (7.24 ppm) and CD₃CN (1.96 ppm). The ¹⁹F chemical shifts are relative to CFCl₃ and arise for the internal standards C₆F₆ (−162.9 ppm) or C₆H₅CF₃ (−63.9 ppm). The ³¹P chemical shifts are relative to H₃PO₄ (85%).

CHNS elemental analyses are carried out in a HEKAtech EA 3000 elemental analyser. A Sartorius M2P electronic microbalance is used for the sample weights.

Infrared spectra are recorded at room temperature using a Digilab Excalibur FTS 3500 FT-IR spectrometer, with a thermoelectrically cooled DTGS detector comprising deuterated triglycine sulfate. The spectra are measured in the wavenumber range from 4000 cm⁻¹ to 500 cm⁻¹ using the ATR (attenuated total reflection) technique with the Mlracle™ ATR accessory, Pike Technologies. The Raman spectra were measured using a Raman accessory for the FTS 3500 with a Nd-YAG crystal or a Bruker Multi Ram, likewise with a Nd-YAG crystal. The measurement range extends from 70 to 3600 cm⁻¹ (Stockes) or −120 to −3600 cm⁻¹ (anti-Stockes).

The x-ray fluorescence analytical measurements are carried out in an Eagle II μ-Probe x-ray fluorescence spectrometer from Röntgenanalytik Messtechnik GmbH with a nitrogen-cooled silicon-lithium detector from EDAX (Mahwah, USA).

### EXAMPLE 1

### Preparation of iron(III) tris[hydrogen(pentafluoroethyl)-phosphonate]

FeCl₃+3C₂F₅P(O)(OH)₂→Fe[C₂F₅P(O)₂OH]₃+3HCl

Anhydrous iron(III) chloride, FeCl₃ (1.49 g, 9.15 mmol), is mixed with pentafluoroethylphosphonic acid, C₂F₅P(O)(OH)₂ (5.49 g, 27.45 mmol), and ground intensively in an agate mortar under an argon atmosphere. The hydrogen chloride formed is removed by means of a gentle stream of argon. The resultant white solid is transferred into a round-bottomed flask and heated at 140° C. for 20 minutes with stirring. The solid is subsequently dried at 1 kPa and 50° C. for two hours. Iron(III) tris[hydrogen(pentafluoroethyl)phosphonate], Fe[C₂F₅P(O)₂OH]₃ (5.64 g, 8.644 mmol, 94% of the theoretical yield), is isolated as a white solid.

The isolated product is characterised by means of ¹H, ¹⁹F and ³¹P spectra, elemental analysis, vibration spectroscopy and ion chromatography.

NMR (solvent: CD₃OD; δ in ppm),

¹⁹F-NMR: −83.0 s (3F), -128.3 d, ²J_{F,P}=76 Hz, (2F).

³¹P-NMR: −2.3 t, ²J_{P,F}=76 Hz.

NMR (solvent: dimethyl sulfoxide-d6, δ in ppm)

¹H NMR: 7.27 (s, OH).

¹⁹F NMR: −80.6 (s, 3F), −126.0 (d, ²J_{F,P}=73 Hz, 2F).

³¹P NMR: −4.5 (t, ²J_{P,F}=73 Hz).

Elemental analysis:

Theoretical % für C₆H₃F₁₅FeO₉P₃: C, 11.04, H, 0.46.

Experimental %: C, 10.98, H, 0.46.

IR spectrum (cm⁻¹):

3643 (20), 3573 (17), 2800 (9), 1772 (5), 1628 (5), 1326 (33), 1209 (99), 1184 (96), 1167 (98), 1107 (100), 1008 (37), 959 (37), 942 (36), 912 (43), 754 (20), 635 (20), 575 (50).

Raman spectrum (cm⁻¹)

1328 (16), 1181 (83), 1129 (29), 1015 (16), 949 (8), 915 (14), 756 (100), 637 (22), 595 (22), 526 (11), 487 (9), 467 (11), 400 (13), 367 (32), 316 (18), 302 (19), 245 (20), 172 (18), 140 (20), 123 (19), 95 (21).

Ion chromatography:

chloride ions <1 ppm, t (min): phosphonate: 12.25.

Fe(II) detection negative:

Fe[C₂F₅P(O)₂OH]₃ (0.0059 g, 0.009 mmol) is dissolved in H₂O (bidist, 0.581 g), and two drops of a two percent alcoholic solution of 2,2′-bipyridine are added. The solution becomes cloudy and changes colour to pale yellow. After addition of two drops (0.074 mg) of an iron(II) sulfate solution (c=0.0008 mol·l⁻¹) a clear red coloration occurs after 5 minutes.

### EXAMPLE 2

### Preparation of aluminium(III) tris[hydrogen(pentafluoroethyl)phosphonate]

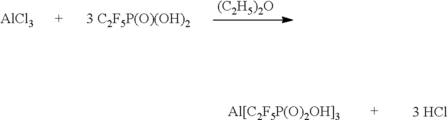

Pentafluoroethylphosphonic acid, (C₂F₅P(O)(OH)₂ (19.25 g, 96.2 mmol), and aluminium trichloride, AlCl₃ (4.27 g, 32.1 mmol), are initially introduced in a 100 ml round-bottomed flask. Diethyl ether (42.67 g, 575.7 mmol) is added to the mixture at 0° C. (ice bath), and the reaction mixture is evaporated to ⅓ of the volume at a bath temperature of 55° C. 45 ml of diethyl ether are added at room temperature and distilled off at a bath temperature of 55° C. This operation is repeated (at least 5 times) until chloride ions are no longer detectable in the distillate (AgNO₃ soln.). The white, viscous crude product is dried at 0.1 Pa, firstly at room temperature (36 h) and subsequently at 120° C. (5 h). Aluminium(III) tris(pentafluoroethylhydrogenphosphonate), Al[C₂F₅P(O)₂OH]₃ (19.39 g, 31.1 mmol, 97% of the theoretical yield), is isolated as a white solid yield.

The isolated product is characterised by means of ¹H, ¹⁹F and ³¹P NMR spectra, x-ray fluorescence analysis, elemental analysis and vibration spectroscopy.

NMR (solvent: 1-butyl-3-methylimidazolium trifluoromethanesulfonate;

CD₃CN film, δ in ppm),

¹H NMR: 10.40 (s, H).

¹⁹F NMR: −82.9 (s, 3F), −128.3 (d, ²J_{F,P}=85 Hz, 2F).

³¹P NMR: −3.8 (t, ²J_{P,F}=85 Hz).

X-ray fluorescence analysis:

Theoretical: Al:P:Cl=1:3:0.

Experimental: Al:P:Cl=1:2.99:0.

Elemental analysis:

Theoretical (%) für AlC₆F₁₅H₃O₉P₃: C, (11.55), H, (0.48).

Experimental (%): C, (11.49), H, (0.45).

IR spectrum (cm⁻¹):

3666 (12), 1635 (17), 1530 (14), 1450 (13), 1392 (12), 1324 (38), 1213 (100), 1168 (76), 1121 (83), 991 (53), 953 (54), 828 (23), 777 (21), 754 (32), 635 (21), 577 (43).

Raman spectrum (cm⁻¹):

1328 (10), 1239 (12), 1222 (10), 1204 (8), 1132 (4), 1117 (5), 1016 (6), 914 (10), 756 (100), 637 (15), 597 (13), 534 (6), 475 (4), 367 (10), 342 (3), 302 (5), 241 (6), 181 (3), 109 (1).

### EXAMPLE 3

### Preparation of copper (II) bis[hydrogen(pentafluoroethyl)-phosphonate]

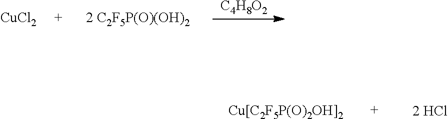

Pentafluoroethylphosphonic acid, (C₂F₅P(O)(OH)₂ (3.75 g, 18.75 mmol), and copper(II) chloride, (1.26 g, 9.38 mmol), are initially introduced in a 100 ml round-bottomed flask. 100 ml of 1,4-dioxane are added at room temperature and distilled off at a bath temperature of 135° C. This operation is repeated (at least 20 times) until chloride ions are no longer detectable in the distillate (AgNO₃ soln.). The grey crude product is dried at 0.1 Pa and 140° C. for 20 h, subsequently washed twice with 10 ml of dichloromethane and dried again at 0.1 Pa and 140° C. for 20 h. A chloride- and 1,4-dioxane-containing copper(II) bis(pentafluoroethylhydrogenphosphonate), Cu[C₂F₅P(O)₂OH]₂ (2.81 g, 6.09 mmol, 65% of the theoretical yield), are isolated as a grey solid.

The isolated product is characterised by means of ¹H, ¹⁹F and ³¹P NMR spectra, X-ray fluorescence analysis, elemental analysis and vibration spectroscopy.

NMR (solvent: 1-butyl-3-methylimidazolium trifluoromethanesulfonate;

CD₃CN film, δ in ppm),

¹H NMR: 12.13 (s, OH), 3.16 (s, CH₂).

¹⁹F NMR: −82.9 (s, 3F), −128.3 (d, ²J_{F,P}=84 Hz, 2F).

³¹P NMR: −3.8 (t, ²J_{P,F}=84 Hz).

X-ray fluorescence analysis

Theoretical for Cu[C₂F₅P(O)₂OH]_{1.58}Cl_{0.42}.2 C₄H₈O₂

or Cu₁₂C₄₆H₃₅F₉₅O₄₂P₁₉C₁₅: Cu:P:Cl=1:1.58:0.42.

Experimental: Cu:P:Cl=1:1.51:0.37.

Elemental analysis

Theoretical (%) for Cu[C₂F₅P(O)₂OH]_{1.58}Cl_{0.42}.2 C₄H₈O₂ or Cu₁₂C₄₆H₃₅F₉₅O₄₂P₁₉C₁₅: C, (11.28), H, (0.72).

Experimental (%): C, (11.05), H, (0.82).

IR spectrum (cm⁻¹):

3641 (37), 3402 (29), 1616 (26), 1323 (43), 1212 (100), 1158 (99), 1110 (83), 1005 (61), 957 (64), 928 (67), 873 (31), 752 (36), 633 (31), 588 (37), 576 (44).

### EXAMPLE 4

### Preparation of zinc(II) bis[hydrogen(pentafluoroethyl)-phosphonate]

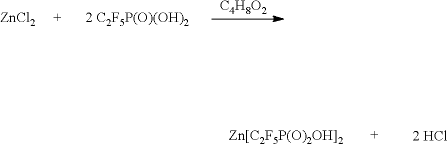

Pentafluoroethylphosphonic acid, (C₂F₅P(O)(OH)₂ (3.70 g, 18.5 mmol), and zinc(II) chloride (1.26 g, 9.3 mmol), are initially introduced in a 100 ml round-bottomed flask. 100 ml of 1,4-dioxane are added at room temperature and distilled off a bath temperature of 135° C. This operation is repeated (at least 15 times) until chloride ions are no longer detectable in the distillate (AgNO₃ soln.). The crude product changes colour to brown in the process. The brown, highly viscous mass dried at 0.1 Pa and 140° C. for 20 h, subsequently washed twice with 10 ml of dichloromethane and dried again in vacuo. A chloride- and 1,4-dioxane-containing zinc(II) bis(pentafluoroethylhydrogenphosphonate), Zn[C₂F₅P(O)₂OH]₂ (3.28 g, 6.75 mmol, 73% of the theoretical yield) are isolated as a grey solid.

The isolated product is characterised by means of ¹H, ¹⁹F and ³¹P NMR spectra, x-ray fluorescence analysis, elemental analysis and vibration spectroscopy.

NMR (solvent: CD₃CN; δ in ppm),

¹H NMR: 10.41 (s, OH), 3.64 (s, CH₂).

¹⁹F NMR: −82.9 (s, 3F), −128.3 (d, ²J_{F,P}=85 Hz, 2F).

³¹P NMR: −3.5 (t, ²J_{P,F}=85 Hz).

X-ray fluorescence analysis:

Theoretical for Zn[C₂F₅P(O)₂OH]₂.0.25 C₄H₈O₂ or Zn₂C₁₀H₈F₂₀O₁₃P₄: Zn:P:Cl=1:2:0.

Experimental: Zn: P:Cl=1:1.98:0.10.

Elemental analysis:

Theoretical (%) for Zn[C₂F₅P(O)₂OH]₂.0.25 C₄H₈O₂ or Zn₂C₁₀H₈F₂₀O₁₃P₄: C, (12.37), H, (0.83).

Experimental (%): C, (12.50), H, (0.78).

IR spectrum (cm⁻¹):

3619 (10), 1622 (11), 1458 (7), 1382 (6), 1325 (38), 1211 (100), 1165 (80), 1115 (92), 1081 (68), 1007 (52), 981 (38), 945 (60), 851 (32), 754 (29), 634 (25), 575 (44).

### EXAMPLE 5

### Preparation of nickel(II) bis[hydrogen(pentafluoroethyl)-phosphonate]

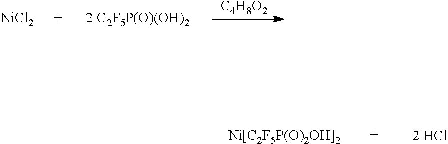

Pentafluoroethylphosphonic acid, (C₂F₅P(O)(OH)₂ (4.04 g, 20.2 mmol), and nickel(II) chloride (1.34 g, 10.3 mmol), are initially introduced in a 100 ml round-bottomed flask. 100 ml of 1,4-dioxane are added at room temperature and distilled off at a bath temperature of 135° C. This operation is repeated (at least 15 times) until chloride ions are no longer detectable in the distillate (AgNO₃ soln.). The crude product changes colour to orange in the process. The orange, highly viscous mass is dried at 0.1 Pa and 140° C. for 10 h, subsequently washed with twice 10 ml of dichloromethane and dried again in vacuo. A chloride- and 1,4-dioxane-containing nickel(II) bis(pentafluoroethylhydrogenphosphonate), Ni[C₂F₅P(O)₂OH]₂ (4.22 g, 9.24 mmol, 91% of the theoretical yield) is isolated as an orange solid. The isolated product is characterised by means of ¹H, ¹⁹F and ³¹P NMR spectra, x-ray fluorescence analysis, elemental analysis and vibration spectroscopy.

NMR (solvent: D₂O; δ in ppm),

¹⁹F NMR: −81.1 (s, 3F), −126.0 (d, ²J_{F,P}=75 Hz, 2F).

³¹P NMR: −2.3 (s, ²J_{P,F}=160 Hz).

IR spectrum (cm⁻¹):

3659 (11), 1623 (9), 1613 (9), 1540 (6), 1459 (7), 1324 (40), 1216 (100), 1166 (83), 1115 (73), 1075 (57), 1003 (53), 934 (49), 872 (35), 753 (28), 633 (27), 576 (50).

### EXAMPLE 6

### Preparation of yttrium(III) tris[hydrogen(pentafluoroethyl)-phosphonate]

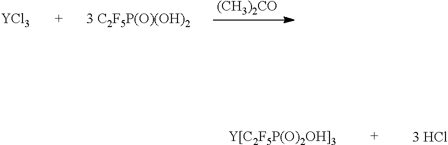

Yttrium(III) chloride, YCl₃ (0.827 g, 4.24 mmol), is dissolved in 35 ml of dry acetone in a 100 ml round-bottomed flask. A solution of pentafluoroethylphosphonic acid, (C₂F₅P(O)(OH)₂ (2.54 g, 12.72 mmol), in 5 ml of acetone is subsequently added. A clear reddish solution is obtained. After addition of a further 30 ml of dry acetone, the solution is stirred at room temperature for 4 hours. The crude product is filtered off as a white solid. The solid is washed with twice with 5 ml of acetone and subsequently dried at 0.1 Pa and 50° C. (6 h). An acetone-containing yttrium(III) tris[hydrogen(pentafluoroethyl)phosphonate], Y[C₂F₅P(O)₂OH]₃ (2.03 g, 2.96 mmol, 70% of the theoretical yield), is isolated as a white solid.

The isolated product is characterised by means of ¹H, ¹⁹F and ³¹P NMR spectra, elemental analysis, vibration spectroscopy and ion chromatography.

NMR (solvent: 1-butyl-3-methylimidazolium trifluoromethanesulfonate; CD₃CN film, δ in ppm)

¹H NMR: 11.08 (s, OH), 2.11 (s, (CH₃)₂CO); intensity ratio 1:1.

¹⁹F NMR: −82.9 (s, 3F), −128.3 (d, ²J_{F,P}=85 Hz, 2F).

³¹P NMR: −3.9 (t, ²J_{P,F}=85 Hz).

Elemental analysis

Theoretical (%) for Y[C₂F₅P(O)₂OH]₃.0.5 (CH₃)₂CO or Y₂C₁₅H₁₂F₃₀O₁₉P₆: C, (12.60), H, (0.85).

Experimental (%): C, (12.93), H, (1.06).

IR spectrum (cm⁻¹)

3691 (3), 3676 (3), 3576 (4), 2930 (10), 2340 (4), 1669 (19), 1425 (3), 1375 (4), 1326 (26), 1215 (100), 1168 (49), 1109 (78), 1005 (20), 944 (32), 752 (10), 633 (13), 581 (38), 562 (10).

Raman spectrum (cm⁻¹)

2936 (18), 1577 (18), 1428 (24), 1327 (37), 1221 (44), 1180 (54), 1113 (34), 1014 (23), 947 (31), 802 (10), 754 (100), 634 (30), 596 (31), 528 (25), 457 (14), 374 (35), 328 (35), 295 (37), 248 (17), 155 (20), 80 (32).

Ion chromatography

chloride ions <1 ppm, t (min): phosphonate: 12.25.

### EXAMPLE 7

### Preparation of bismuth(III) tris[hydrogen(pentafluoroethyl)-phosphonate]

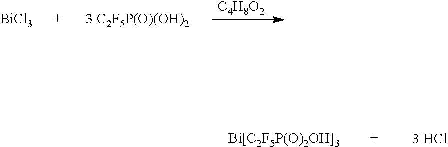

Pentafluoroethylphosphonic acid, (C₂F₅P(O)(OH)₂ (1.51 g, 7.56 mmol), and bismuth(III) chloride, (0.796 g, 2.52 mmol), are initially introduced in a 100 ml round-bottomed flask. 100 ml of 1,4-dioxane are added at room temperature and distilled off at a bath temperature of 135° C. This operation is repeated (at least 20 times) until chloride ions are no longer detectable in the distillate (AgNO₃ soln.). The grey product is dried at 0.1 Pa and 130° C. for 20 h. Bismuth(III) tris(pentafluoroethylhydrogenphosphonate), Bi[C₂F₅P(O)₂OH]₃ (1.73 g, 2.15 mmol, 85% of the theoretical yield), is isolated as a grey solid.

The isolated product is characterised by means of ¹H, ¹⁹F and ³¹P NMR spectra, X-ray fluorescence analysis, elemental analysis and vibration spectroscopy.

NMR (solvent: 1-butyl-3-methylimidazolium trifluoromethanesulfonate; CD₃CN film, δ in ppm),

¹H NMR: 10.73 (s, OH).

¹⁹F NMR: −82.9 (s, 3F), −128.3 (d, ²J_{F,P}=85 Hz, 2F).

³¹P NMR: −3.9 (t, ²J_{P,F}=85 Hz).

NMR (solvent: dimethyl sulfoxide-d6, δ in ppm),

¹H NMR: 12.41 (s, OH).

¹⁹F NMR: −80.4 (s, 3F), −125.3 (d, ²J_{F,P}=85 Hz, 2F).

³¹P NMR: −6.4 (t, ²J_{P,F}=85 Hz).

X-ray fluorescence analysis:

Theoretical for Bi[C₂F₅P(O)₂OH]₃: Bi:P:Cl=1:3:0.

Experimental: Bi: P:Cl=1:1.81:0.00.

Elemental analysis:

Theoretical (%) for Bi[C₂F₅P(O)₂OH]₃: C, (8.94), H, (0.38).

Experimental (%): C, (9.16), H, (0.38).

IR spectrum (cm⁻¹):

3613 (13), 3593 (13), 1608 (9), 1324 (41), 1213 (85), 1160 (96), 1127 (90), 1062 (100), 1009 (69), 957 (69), 929 (65), 913 (57), 754 (38), 634 (26), 573 (42).

### COMPARATIVE EXAMPLE 8A

### Uncatalysed Michael Reaction of methyl vinyl ketone and (1-methoxy-2-methylprop-1-enyloxy)trimethylsilane

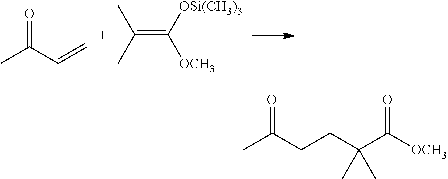

Methyl vinyl ketone, (0.564 g, 8.047 mmol) and (1-methoxy-2-methylprop-1-enyloxy)trimethylsilane, (1.58 g, 9.047 mmol) are combined in a 50 ml two-necked flask, fitted with reflux condenser and drying tube, and dissolved in dichloromethane (13.36 g). The reaction solution is investigated by ¹H-NMR spectroscopy and then stirred at room temperature for 30 hours. The reaction solution is then investigated again by ¹H-NMR spectroscopy, with no change compared with the first measurement being evident. The compounds detected at the end point with reference to the ¹H-NMR spectrum are the unchanged starting materials (1-methoxy-2-methylprop-1-enyloxy)-trimethylsilane and methyl vinyl ketone having the following shift values:

(1-Methoxy-2-methylprop-1-enyloxy)trimethylsilane:

¹H-NMR (solvent: CD₃CN; δ in ppm): 3.47 (s, 3H), 1.55 (s, 3H), 1.51 (s, 3H), 0.20 (s, 9H).

Methyl vinyl ketone:

¹H-NMR (solvent: CD₃CN; δ in ppm): 6.28 (m, 2H), 5.94 (d, ³J_{H,H}=10.1 Hz, 1H), 2.26 (s, 3H).

### EXAMPLE 8B

### Michael Reaction of methyl vinyl ketone and (1-methoxy-2-methylprop-1-enyloxy)trimethylsilane Catalysed by iron(III) tris-[hydrogen(pentafluoroethyl)phosphonate] (10 mol %)

Iron(III) tris[hydrogen(pentafluoroethyl)phosphonate], Fe[(C₂F₅)P(O)₂OH]₃ (0.388 g, 0.594 mmol), is suspended in dichloromethane (13.49 g) in a 50 ml two-necked flask with reflux condenser and drying tube. Methyl vinyl ketone (0.432 g, 6.16 mmol) and (1-methoxy-2-methylprop-1-enyloxy)-trimethylsilane (1.58 g, 9.04 mmol) are added to this suspension. After a reaction time of 3 hours, the conversion to methyl 2,2-dimethyl-5-oxohexenoate, determined using ¹H-NMR spectroscopy, is 98 mol %. The solvent dichloromethane is removed at 0.1 Pa and room temperature. Water (20 ml) is added to the residue, and the resultant emulsion is extracted three times with 20 ml of n-hexane. The catalyst remains in the aqueous phase. After evaporation of the combined n-hexane solutions, methyl 2,2-dimethyl-5-oxohexenoate (0.88 g, 5.11 mmol, 83% of the theoretical yield) can be isolated as a colourless liquid.

Methyl 2, 2-dimethyl-5-oxohexenoate:

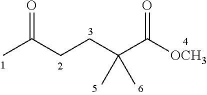

¹H-NMR (solvent: CD₃CN; δ in ppm): 3.63 (s, C⁴H, 3H), 2.41 (m, C²H, 2H), 2.09 (s, C¹H, 3H), 1.74 (m, C³H, 2H), 1.16 (s, C^{5,6}H, 6H).

### EXAMPLE 8C

### Reaction from Example 8b Catalysed by iron(II) tris-[hydrogen(pentafluoroethyl)phosphonate] (4.95 mol %)

Iron(III) tris[hydrogen(pentafluoroethyl)phosphonate], Fe[(C₂F₅)P(O)₂OH]₃ (0.379 g, 0.581 mmol), is suspended in methyl vinyl ketone (0.432 g, 6.16 mmol) in a 50 ml two-necked flask with reflux condenser and drying tube. (1-Methoxy-2-methylprop-1-enyloxy)trimethylsilane (3.09 g, 17.70 mmol) is added to this suspension. The suspension warms. After a reaction time of 1 hour, water (20 ml) is added to the solution, and the resultant emulsion is extracted three times with 20 ml of n-hexane. After evaporation of the hexane and the (1-methoxy-2-methylprop-1-enyloxy-trimethylsilane employed in excess, methyl 2,2-dimethyl-5-oxohexenoate (1.50 g, 8.69 mmol, 74% of the theoretical yield) can be isolated as a colourless liquid at 90° C. and 0.1 Pa.

¹H-NMR (solvent: CD₃CN; δ in ppm): 3.62 (s, C⁴H, 3H), 2.40 (m, C²H, 2H), 2.08 (s, C¹H, 3H), 1.74 (m, C³H, 2H), 1.15 (s, C^{5,6}H, 6H).

### EXAMPLE 8D

### Solvent-Free Michael Reaction of methyl vinyl ketone and (1-methoxy-2-methylprop-1-enyloxy)trimethylsilane Catalysed by aluminium(III) tris[hydrogen(pentafluoroethyl)phosphonate](4.95 mol %)

Aluminium(III) tris[hydrogen(pentafluoroethyl)phosphonate], Al[(C₂F₅)P(O)₂OH]₃ (0.259 g, 0.415 mmol), is suspended in (1-methoxy-2-methylprop-1-enyloxy)trimethylsilane (1.48 g, 8.51 mmol) in a 50 ml two-necked flask with reflux condenser and drying tube. Methyl vinyl ketone (0.586 g, 8.36 mmol) is added to this suspension. The suspension warms. After a reaction time of 5 minutes, water (0.3 ml) is added to the suspension. The suspension is filtered, and the water is removed in vacuo at room temperature. Methyl 2,2-dimethyl-5-oxohexenoate (0.81 g, 4.73 mmol, 57% of the theoretical yield) can be isolated as a colourless liquid at 90° C. and 0.1 Pa.

¹H-NMR (solvent: CD₃CN; δ in ppm): 3.62 (s, C⁴H, 3H), 2.41 (m, C²H, 2H), 2.08 (s, C¹H, 3H), 1.74 (m, C³H, 2H), 1.15 (s, C^{5,6}H, 6H).

### COMPARATIVE EXAMPLE 9A

### Uncatalysed, Solvent-Free Michael Reaction of 2-cyclohexen-1-one and (1-methoxy-2-methylprop-1-enyloxy)-trimethylsilane

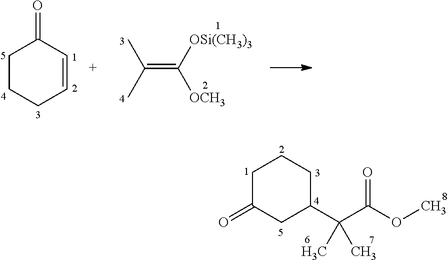

2-Cyclohexen-1-one (0.533 g, 5.545 mmol) and (1-methoxy-2-methylprop-1-enyloxy)trimethylsilane (1.05 g, 6.029 mmol) are mixed in a 50 ml two-necked flask and stirred at room temperature. The reaction solution is investigated by ¹H-NMR spectroscopy and then stirred at room temperature for 24 hours. The reaction solution is subsequently investigated again by ¹H-NMR spectroscopy, with no change being evident.

The compounds detected at the endpoint with reference to the ¹H-NMR spectrum are the unchanged starting materials (1-methoxy-2-methylprop-1-enyloxy)trimethylsilane and 2-cyclohexen-1-one having the following shift values.

(1-Methoxy-2-methylprop-1-enyloxy)trimethylsilane:

¹H-NMR (solvent: CD₃CN; δ in ppm): 3.48 (s, C²H, 3H), 1.55 (s, C³H, 3H), 1.51 (s, C⁴H, 3H), 0.20 (s, C⁴H, 9H).

2-Cyclohexen-1-one:

¹H-NMR (solvent: CD₃CN; δ in ppm): 7.06 (m, C²H, 1H), 5.93 (d, C¹H, 1H), 2.36 (m, C^{3,5}H, 4H), 1.99 (m, C⁴H, 2H).

### EXAMPLE 9B

### Solvent-Free Michael Reaction of 2-cyclohexen-1-one and (1-methoxy-2-methylprop-1-enyloxy)trimethylsilane Catalysed by iron(II) tris[hydrogen(pentafluoroethyl)phosphonate] (2 mol %)

Iron(III) tris[hydrogen(pentafluoroethyl)phosphonate], Fe[(C₂F₅)P(O)₂OH]₃ (0.139 g, 0.21 mmol) is dissolved in 2-cyclohexen-1-one (1.03 g, 10.66 mmol) in a 50 ml two-necked flask with reflux condenser and drying tube. (1-Methoxy-2-methylprop-1-enyloxy)trimethylsilane (1.87 g, 10.71 mmol) is added to this solution. After a reaction time of 24 hours, a conversion of 52 mol % can be determined by ¹H-NMR spectroscopy. 30 ml of water are added to the reaction mixture, which is then extracted with 30 ml of dichloromethane. The organic phase is separated off, and the dichloromethane is removed in vacuo at 0.1 Pa and room temperature, giving methyl 2-methyl-2-(3-oxocyclohexyl)propanoate (1.08 g, 5.442 mmol, 51% of the theoretical yield) as a white solid.

Methyl 2-methyl-2-(3-oxocyclohexyl)propanoate:

¹H-NMR (solvent: CD₃CN; δ in ppm): 3.63 (s, C⁸H, 3H), 2.37 (m, C^{1,4,5,5′}H, 4H), 2.01 (s, C^{1′,2}H, 2H), 1.86 (m, C^{2′}H, 1H), 1.54 (m, C³H, 1H), 1.30 (m, C^{3′}H, 1H), 1.15 (s, C⁶H, 3H), 1.16 (s, C⁷H, 3H).

1H-NMR (CDCl₃; δ in ppm): 3.68 (s, 3H), 2.43-1.96 (m, 6H), 1.84-1.71 (m, 1H), 1.68-1.50 (m, 1H), 1.46-1.30 (m, 1H), 1.17 (s, 3H), 1.15 (s, 3H).]

The NMR spectra correspond to the literature data [R. Nagase, J. Osada, H. Tamagaki, Y. Tanabe, Adv. Synth. Catal. 2010, vol. 352, pp. 1128-1134].

### EXAMPLE 9C

### Solvent-Free Michael Reaction of 2-cyclohexen-1-one and (1-methoxy-2-methylprop-1-enyloxy)trimethylsilane Catalysed by iron(II) tris[hydrogen(pentafluoroethyl)phosphonate] (10 mol %)

Iron(III) tris[hydrogen(pentafluoroethyl)phosphonate], Fe[(C₂F₅)P(O)₂OH]₃ (0.161 g, 0.247 mmol) is dissolved in 2-cyclohexen-1-one (0.239 g, 2.486 mmol) in a 50 ml two-necked flask with reflux condenser and drying tube. (1-Methoxy-2-methylprop-1-enyloxy)trimethylsilane (0.469 g, 2.691 mmol) is added to this solution. After a reaction time of 24 hours, a conversion of 69 mol % can be determined by means of ¹H-NMR spectroscopy. 30 ml of water are added to the reaction mixture, which is then extracted three times with 10 ml of n-hexane. The organic phases are in each case separated off, combined, and the n-hexane is removed at 0.1 Pa and room temperature. Methyl 2-methyl-2-(3-oxocyclohexyl)propanoate (0.245 g, 1.236 mmol, 50% of the theoretical yield) is isolated as a white solid. The product is characterised by means of ¹H-NMR spectroscopy. The chemical shifts and coupling values correspond to the shift values indicated in Example 9b.

### COMPARATIVE EXAMPLE 10A

### Uncatalysed, Solvent-Free Michael Reaction of methyl vinyl ketone and acetylacetone

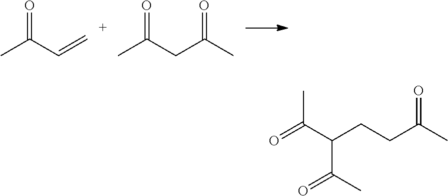

Methyl vinyl ketone (0.511 g, 7.291 mmol) and acetylacetone (0.785 g, 7.841 mmol) are mixed in a 50 ml two-necked flask and stirred at room temperature. After a reaction time of 24 hours, a conversion of 75% can be determined by ¹H-NMR spectroscopy. The compounds detected at the end point with reference to the ¹H-NMR spectrum are acetylacetone, methyl vinyl ketone, 3-acetylheptane-2,6-dione (keto form) and 3-(1-hydroxyethylidene)heptane-2,6-dione (enol form) having the following shift values. The ratio of methyl vinyl ketone to the tautomers 3-acetylheptane-2,6-dione (keto form) and 3-(1-hydroxyethylidene)heptane-2,6-dione (enol form) is 25:55:20.

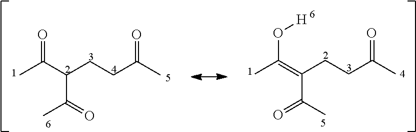

Acetylacetone (keto/enol form):

¹H-NMR (solvent: CD₃CN; δ in ppm): 2.04 (m, enol CH₃, 6H), 2.17 (s, keto CH₃, 6H), 3.62 (s, enol CH, 1H), 5.65 (s, keto CH, 1H), 15.61 (s, enol OH, 1H).

Methyl vinyl ketone:

¹H-NMR (solvent: CD₃CN; δ in ppm): 6.28 (m, C^{2,3}H, 2H), 5.94 (d, ³J_{H,H}=10.1 Hz, C³H, 1H), 2.26 (s, C^{1,1′}H, 6H).

3-Acetylheptane-2,6-dione (keto form):

¹H-NMR (solvent: CD₃CN; δ in ppm): 3.76 (t, ³J_{H,H}=6 Hz, C²H, 1H), 2.43 (t, ³J_{H,H}=6.0 Hz, C³H, 2H), 2.07 (s, C^{1,6}H, 6H), 2.04 (m, C⁵H, 3H), 2.00 (m, C⁴H, 2H).

The NMR spectra correspond to the literature data [M. Picquet, C. Bruneau, P. H. Dixneuf, Tetrahedron, 1999, vol. 55, p. 3937]:

¹H-NMR (solvent: CDCl₃, δ in ppm): 3.62 (t, ³J_{H,H}=7 Hz, 1H), 2.33 (t, ³J_{H,H}=7 Hz, 2H), 2.07 (s, 6H), 2.00 (s, 3H) 2.05-1.87 (m, 2H).]

3-(1-Hydroxyethylidene)heptane-2,6-dione (enol form)

¹H-NMR (solvent: CD₃CN; δ in ppm): 16.83 (s, OH, 1H), 2.53 (dm, C³H, 2H), 2.22 (dm, C²H, 2H), 2.16 (s, C^{4,5}H, 6H), 2.13 (s, C¹H, 3H).

The NMR spectra correspond to the literature data [C. Allais, F. Liéby-Muller, J. Rodriguez, T. Constantieux, Eur. J. Org. Chem., 2013, pp. 4131-4145]: ¹H-NMR data for both tautomers; solvent: CDCl₃, δ in ppm: 15.73 (br s, 1H), 3.61 (t, J=6.9 Hz, 1H), 2.50-2.45 (m, 2H), 2.37 (d, J=7.1 Hz, 2H), 2.11 (s, 9H), 2.09 (s, 3H), 2.06 (s, 3H), 2.04 (s, 3H), 2.02-1.95 (m, 4H).

### EXAMPLE 10B

### Solvent-Free Michael Reaction of methyl vinyl ketone with acetylacetone Catalysed by iron(III) tris[hydrogen(pentafluoroethyl)phosphonate] (10 mol %)

Iron(III) tris[hydrogen(pentafluoroethyl)phosphonate], Fe[C₂F₅P(O)₂OH]₃ (0.252 g, 0.386 mmol), is initially introduced in a 50 ml two-necked flask, and methyl vinyl ketone (0.279 g, 3.981 mmol) and acetylacetone (0.617 g, 6.163 mmol) are subsequently added and the mixture is stirred at room temperature. After a reaction time of 5 minutes, complete conversion of methyl vinyl ketone into the product can be detected by means of ¹H-NMR spectroscopy.

The compounds detected at the end point with reference to the ¹H-NMR spectrum are acetylacetone, 3-acetylheptane-2,6-dione (keto form) and 3-(1-hydroxyethylidene)heptane-2,6-dione (enol form) with the shift values indicated in Example 9a. The ratio of the tautomers 3-acetylheptane-2,6-dione (keto form) and 3-(1-hydroxyethylidene)heptane-2,6-dione (enol form) is 65:35.

### EXAMPLE 10C

### Solvent-Free Michael Addition of methyl vinyl ketone and acetylacetone Catalysed by iron(III) tris[hydrogen(pentafluoroethyl)-phosphonate] (2 mol %)

Iron(III) tris[hydrogen(pentafluoroethyl)phosphonate], Fe[C₂F₅P(O)₂OH]₃ (0.063 g, 0.097 mmol), is initially introduced in a 50 ml two-necked flask, and methyl vinyl ketone (0.360 g, 5.136 mmol) and acetylacetone (0.779 g, 7.781 mmol) are subsequently added, and the mixture is stirred at room temperature. After a reaction time of one hour, complete conversion of methyl vinyl ketone to the product can be detected by means of ¹H-NMR spectroscopy.

The compounds detected at the end point with reference to the ¹H-NMR spectrum are acetylacetone, 3-acetylheptane-2,6-dione (keto form) and 3-(1-hydroxyethylidene)heptane-2,6-dione (enol form) having the shift values indicated in Example 9a. The ratio of the tautomers 3-acetylheptane-2,6-dione (keto form) and 3-(1-hydroxyethylidene)heptane-2,6-dione (enol form) is 67:33.

### EXAMPLE 10D

### Reaction from Example 10c with a Larger Excess of Acetylacetone

Iron(III) tris[hydrogen(pentafluoroethyl)phosphonate], Fe[C₂F₅P(O)₂OH]₃ (0.102 g, 0.156 mmol), is initially introduced in a 50 ml two-necked flask, and methyl vinyl ketone (0.582 g, 8.304 mmol) and acetylacetone (1.93 g, 19.317 mmol) are subsequently added, and the mixture is stirred at room temperature. After a reaction time of four hours, complete conversion of methyl vinyl ketone to the product can be detected by means of ¹H-NMR spectroscopy.

The compounds detected at the end point with reference to the ¹H-NMR spectrum are acetylacetone, 3-acetylheptane-2,6-dione (keto form) and 3-(1-hydroxyethylidene)heptane-2,6-dione (enol form) having the shift values indicated in Example 9a. Increasing the amount of acetylacetone to 2.5 times the amount enables a ratio of the tautomers 3-acetylheptane-2,6-dione (keto form) and 3-(1-hydroxyethylidene)heptane-2,6-dione (enol form) of 53:47 to be obtained.

### COMPARATIVE EXAMPLE 11A

### Uncatalysed, Solvent-Free Michael Reaction of 2-cyclohexen-1-one and acetylactetone

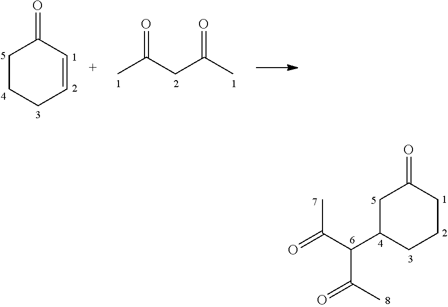

2-Cyclohexen-1-one (0.571 g, 5.940 mmol) and acetylacetone (0.664 g, 6.632 mmol) are combined in a 50 ml two-necked flask. The reaction solution is investigated by ¹H-NMR spectroscopy and then stirred at room temperature for 24 hours. The reaction solution is then investigated again by ¹H-NMR spectroscopy, with no change being evident.

The compounds detected at the end point with reference to the ¹H-NMR spectrum are the unchanged starting materials acetylacetone and 2-cyclohexen-1-one having the following shift values.

Acetylacetone (keto/enol form):

¹H-NMR (solvent: CD₃CN; δ in ppm): 2.04 (m, enol CH₃, 6H), 2.16 (s, keto CH₃, 6H), 3.62 (s, enol CH, 1H), 5.63 (s, keto CH, 1H), 15.62 (s, enol OH, 1H).

2-Cyclohexen-1-one:

¹H-NMR (solvent: CD₃CN; δ in ppm): 7.06 (dt, ³J_{H,H}=10.1 Hz, ³J_{H,H}=4.1 Hz, C²H, 1H), 5.93 (dt, C¹H, ³J_{H,H}=10.1 Hz, ⁴J_{H,H}=1.9 Hz, 1H), 2.36 (m, C^{3,5}H, 4H), 2.00 (m, C⁴H, 2H).

### EXAMPLE 11B

### Solvent-Free Michael Reaction of 2-cyclohexen-1-one and acetylacetone Catalysed by iron(III) tris[hydrogen(pentafluoroethyl)-phosphonate] (2 mol %)

Iron(III) tris[hydrogen(pentafluoroethyl)phosphonate], Fe[C₂F₅P(O)₂OH]₃ (0.089 g, 0.136 mmol), is initially introduced in a 50 ml two-necked flask, and 2-cyclohexen-1-one (0.658 g, 6.845 mmol) and acetylacetone (1.37 g, 13.654 mmol) are subsequently added, and the mixture is stirred at room temperature. After 3 days, a conversion of 97 mol % from 2-cyclohexen-1-one to 3-(3-oxocyclohexyl)pentane-2,4-dione can be determined by ¹H-NMR. The acetylacetone is removed from the reaction mixture at room temperature and 0.1 Pa. Extraction with n-hexane (10×10 ml) enables 3-(3-oxocyclohexyl)pentane-2,4-dione (0.456 g, 2.325 mmol, 34% of the theoretical yield) to be obtained as colourless crystals, having the following shift values:

3-(3-Oxocyclohexyl)pentane-2,4-dione:

¹H-NMR (solvent: CD₃CN; δ in ppm): 1.44 (m, 1H), 1.66 (m, 2H), 2.02 (m, 1H), 2.15 (m, 1H), 2.29 (m, 2H), 2.55 (m, 1H), 3.85 (d, ³J_{H,H}=9.5 Hz, 1H). GC-MS: tR 7.11 min, MS (m/z) (rel. abund.): 196 (M+) (1), 153 (52), 136 (8), 125 (12), 111 (52), 97 (100), 85 (24), 68 (20), 55 (16), 43 (100).

The NMR spectrum corresponds to the literature data [A. Oge, M. E. Mavis, C. Yolacan, F. Aydogan, Turk. J. Chem., 2012, vol. 36, p. 137]:

¹H-NMR (solvent: CDCl₃; δ in ppm): 1.30-1.40 (m, 1H, CH₂), 1.62-1.74 (m, 1H, CH₂), 1.75-1.82 (m, 1H, CH₂), 1.98-2.06 (m, 2H, CH₂), 2.14 (s, 3H, CH₃), 2.16 (s, 3H, CH₃), 2.20-2.31 (m, 2H, CH₂), 2.35-2.41 (m, 1H, CH₂), 2.61-2.71 (m, 1H, CHCH₂), 3.61 (d, J=10.4 Hz, 1H, CH(COCH₃)₂).

GC-MS: tR 18.08 min, MS (m/z) (rel. abund.): 196 (M+) (1), 153 (43), 111 (23), 97 (57), 43 (100).]

### COMPARATIVE EXAMPLE 12A

### Uncatalysed Friedel-Crafts Acylation of Anisole Using Acetic Anhydride

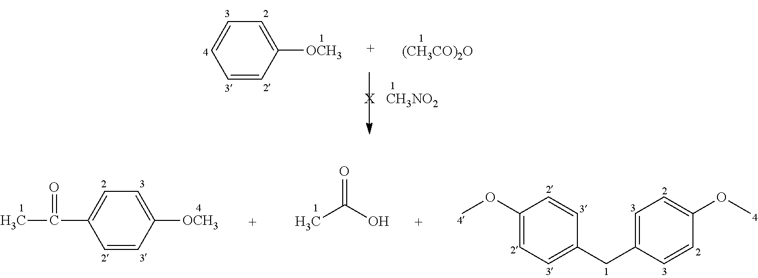

Nitromethane (7.68 g, 0.125 mol) and anisole (0.350 g, 3.237 mmol) are stirred in a 50 ml two-necked flask at an oil-bath temperature of 80° C. for 4.5 h. Conversion to bis(p-methoxyphenyl)methane cannot be determined by ¹H-NMR spectroscopy. In the next step, acetic anhydride (0.462 g, 4.490 mmol) is added to the reaction solution. The reaction solution is investigated by ¹H-NMR spectroscopy and then stirred at an oil-bath temperature of 80° C. for 4.5 hours. The reaction solution is subsequently investigated again by ¹H-NMR spectroscopy, with no change being evident. The compounds detected at the end point with reference to the ¹H-NMR spectrum are the unchanged starting materials acetic anhydride, anisole and nitromethane having the following shift values.

Acetic anhydride:

¹H-NMR (solvent: CD₃CN; δ in ppm): 2.17 (s, C¹H, 6H).

Anisole:

¹H-NMR (solvent: CD₃CN; δ in ppm): 7.32 (m, C^{3,3′,4}H, 3H), 6.96 (m, C^{2,2′}H, 2H), 3.80 (s, C¹H, 3H).

Nitromethane:

¹H-NMR (solvent: CD₃CN; δ in ppm): 4.33 (s, C¹H, 3H).

### EXAMPLE 12B

### Friedel-Crafts Acylation of Anisole Using acetic anhydride Catalysed by iron(III) tris[hydrogen(pentafluoroethyl)-phosphonate] (20 mol %)

Iron(III) tris[hydrogen(pentafluoroethyl)phosphonate], Fe[(C₂F₅)P(O)₂OH](0.384 g, 0.588 mmol), and nitromethane (8.07 g) are combined with anisole (0.326 g, 3.015 mmol) and acetic anhydride (0.614 g, 6.014 mmol) in a 50 ml two-necked flask and stirred at an oil-bath temperature of 80° C. for 90 minutes. The conversion is determined by ¹H-NMR spectroscopy and is 99 mol %. The solvent nitromethane is removed from the reaction mixture at 0.1 Pa and room temperature. 20 ml of a 5% NaHCO₃ solution are added to the reaction mixture. The product is extracted with diethyl ether (3×10 ml). The combined ether phases are evaporated to dryness at room temperature and 0.1 Pa and subsequently extracted twice with 10 ml of n-hexane. Evaporation of the combined hexane phases to dryness at 0.1 Pa and room temperature gives 4-methoxyacetophenone, (0.311 g, 2.071 mmol, 68.7% of the theoretical yield) as a white solid. The product contains 5% of bis(p-methoxyphenyl)methane) impurity. 4-Methoxyacetophenone:

¹H-NMR (solvent: CDCl₃; δ in ppm): 7.96 (m, C^{2,2′}H, 2H), 7.02 (m, C^{3,3′}H, 2H), 3.88 (s, C¹H, 3H), 2.53 (s, C^{4,4′}H, 3H).

¹³C-NMR (solvent: CDCl₃; δ in ppm): 196.8, 163.59, 130.6, 130.4, 113.7, 55.5, 26.3.

The NMR spectra correspond to the data given in the literature [Qian, Adv. Synth. Catal., 2012, vol. 354, p. 3231]:

¹H NMR (300 MHz, CDCl₃) δ: 7.92 (d, J=6.6 Hz, 2H), 6.91 (d, J=6.6 Hz, 2H), 3.85 (s, 3H), 2.54 (s, 3H); ¹³C NMR (100 MHz, CDCl₃) δ: 196.74, 163.37, 130.49, 130.19, 113.57, 55.37, 26.26]

Bis(p-methoxyphenyl)methane

¹H-NMR (solvent: CDCl₃; δ in ppm): 7.14 (dm, ³J_{H,H}=8.4 Hz, C^{2,2,2′,2′}H, 4H), 6.85 (dm, ³J_{H,H}=8.4 Hz, C^{3,3,3′,3′}H, 4H), 3.89 (s, C¹H, 2H), 3.76 (s, C^{4,4′}H, 6H).

¹³C-NMR (solvent: CDCl₃; δ in ppm): 157.9, 133.7, 129.7, 113.8, 55.2, 40.1. The NMR spectra correspond to the data given in the literature [Chen, Tetrahedron, 2014, vol. 70, p. 1975].

¹H NMR (300 MHz, CDCl₃) δ 7.11 (d, J=8.5 Hz, 4H), 6.84 (d, J=8.6 Hz, 4H), 3.89 (s, 2H), 3.80 (s, 6H).

### EXAMPLE 12C

### Friedel-Crafts Acylation of Anisole Using acetic anhydride Catalysed by iron(III) tris[hydrogen(pentafluoroethyl)-phosphonate] (5 mol %)

Iron(III) tris[hydrogen(pentafluoroethyl)phosphonate], Fe[(C₂F₅)P(O)₂OH](0.099 g, 0.152 mmol), and nitromethane (8.00 g) are combined with anisole (0.328 g, 3.033 mmol) and acetic anhydride (0.619 g, 6.063 mmol) in a 50 ml two-necked flask and stirred at an oil-bath temperature of 80° C. for three hours. The conversion to 4-methoxyacetophenone determined by ¹H-NMR spectroscopy is 81.7 mol %. The compounds detected at the end point with reference to the ¹H-NMR spectrum are 4-methoxyacetophenone, bis(p-methoxyphenyl)methane), acetic anhydride, acetic acid, anisole and nitromethane having the shift values indicated in Example 12a and Example 12b. The molar ratio of anisole to 4-methoxyacetophenone to bis(p-methoxyphenyl)methane is 21.7:75.3:1. 20 ml of H₂O and 20 ml of n-hexane are added to the reaction mixture. On addition of n-hexane, three liquid phases form. The product is extracted with four further 20 ml portions of n-hexane. The n-hexane phase is separated off in each case. The five n-hexane phases are combined and evaporated to dryness at room temperature and 0.1 Pa, giving 4-methoxyacetophenone (0.244 g, 1.624 mmol) as a white solid. 20 ml of chloroform are added to the remaining phases which have already been extracted with n-hexane. The organic phase is separated off and evaporated to dryness at 0.1 Pa and room temperature. Re-extraction with 20 ml of n-hexane and evaporation to dryness enables further 4-methoxyacetophenone (0.079 g, 0.526 mmol) to be obtained as a white solid. The total yield is 0.323 g (2.151 mmol, 71% of the theoretical yield) of 4-methoxyacetophenone (4.5% of bis(p-methoxyphenyl)methane) impurity having the shift values indicated in Example 12b.

### EXAMPLE 12D

### Friedel-Crafts Acylation of Anisole Using acetic anhydride Catalysed by iron(III) tris[hydrogen(pentafluoroethyl)-phosphonate] (2 mol %)

Anisole (0.325 g, 3.005 mmol) and acetic anhydride (0.646 g, 6.328 mmol) are added to 0.039 g (0.060 mmol) of iron(III) tris[hydrogen(pentafluoroethyl)phosphonate], Fe[(C₂F₅)P(O)₂OH]₃, in nitromethane (8.099 g) in a 50 ml two-necked flask and the mixture is stirred at an oil-bath temperature of 80° C. for 4 hours. The conversion to 4-methoxyacetophenone subsequently determined by ¹H-NMR spectroscopy is 77 mol %. The compounds detected at the end point with reference to the ¹H-NMR spectrum are 4-methoxyacetophenone, bis(p-methoxyphenyl)methane), acetic anhydride, acetic acid, anisole and nitromethane having the shift values indicated in Example 12b. The molar ratio of anisole to 4-methoxyacetophenone to bis(p-methoxyphenyl)methane) is 22.8:77.8:2.4. 20 ml of H₂O are added to the reaction mixture. The product is extracted with chloroform (3×10 ml).

The combined chloroform phases are evaporated to dryness at room temperature and 0.1 Pa. 4-Methoxyacetophenone (0.298 g, 1.984 mmol, 66% of the theoretical yield) is isolated as a colourless solid and has the shift values indicated in the ¹H-NMR spectrum in Example 12b (the product contains 7% of bis(p-methoxyphenyl)methane) impurity.

### EXAMPLE 12E

### Reaction from Example 12b with Reversed Sequence of the Addition of the Starting Materials

Acetic anhydride 0.646 g (6.328 mmol) and anisole 0.328 g (3.033 mmol) are added to a suspension of iron(III) tris[hydrogen(pentafluoroethyl)-phosphonate], Fe[(C₂F₅)P(O)₂OH]₃ (0.391 g, 0.599 mmol), in nitromethane (8.40 g) in a 50 ml two-necked flask, and the mixture is stirred at an oil-bath temperature of 80° C. for 90 minutes. The conversion to 4-methoxyacetophenone determined by ¹H-NMR spectroscopy is 99 mol %. 20 ml of H₂O are added to the reaction mixture. The product is extracted with 5×20 ml of n-hexane. The combined n-hexane phases are evaporated to dryness at room temperature and 0.1 Pa. 4-Methoxyacetophenone (0.297 g, 1.978 mmol, 65.2% of the theoretical yield), is isolated as a colourless solid and has the shift values indicated in the ¹H-NMR spectrum in Example 12b. By changing the sequence of addition of the starting materials, the formation of bis(p-methoxyphenyl)methane is prevented.

### COMPARATIVE EXAMPLE 13A

### Uncatalysed Methanolysis of Octyl Acetate

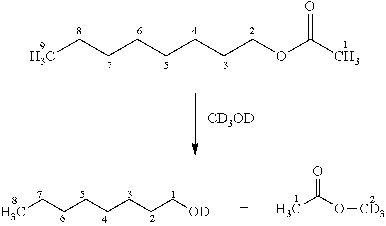

Methanol (0.5 ml) is added to 1-octyl acetate (0.043 ml, 0.290 mmol) in an NMR tube. The reaction solution is investigated by ¹H-NMR spectroscopy and then stirred at an oil-bath temperature of 50° C. for 24 hours. The reaction solution is subsequently investigated again by ¹H-NMR spectroscopy. The conversion determined after a reaction time of 24 hours is less than 1%.

The compounds detected at the end point with reference to the ¹H-NMR spectrum are: 1-octanol, octyl acetate, methyl acetate and methanol having the following shift values, but principally octyl acetat. The molar ratio of octyl acetate to octanol is: 1232:1.

1-Octanol:

¹H-NMR (solvent: CD₃OD; δ in ppm): 4.70 (s, OH, 1H)*, 3.57 (t, ³J_{H,H}=6.6 Hz, C¹H, 2H), 1.56 (m, C²H, 2H), 1.36 (m, C^{3,4′5,6′7}H, 10H), 0.94 (m, C⁸H, 3H).

1-Octyl acetate:

¹H-NMR (solvent: CD₃OD; δ in ppm): 4.10 (t, ³J_{H,H}=6.6 Hz, C2H, 2H), 2.08 (s, C¹H, 3H), 1.67 (m, C³H, 2H), 1.36 (m, C^{4,5,6,7,8}, 10H), 0.94 (m, C⁹H, 3H).

Methyl acetate:

¹H-NMR (solvent: CD₃OD; δ in ppm): 3.35 (s, C²H, 3H)*, 2.07 (m, C¹H, 3H).

Methanol:

¹H-NMR (solvent: CD₃OD; δ in ppm): 4.70 (s, OH, 1H)*, 3.33 (s, CH, 3H)* *(residual protons from the CD₃OD used are detected:

### EXAMPLE 13B

### Methanolysis of octyl acetate Catalysed by yttrium(III) tris[hydrogen(pentafluoroethyl)phosphonate] (10 mol %)

Yttrium(III) tris[hydrogen(pentafluoroethyl)phosphonate], Y[(C₂F₅)₂P(O)₂OH]₃ (3.11 mg*, 0.00453 mmol), is dissolved in CD₃OD (0.5 ml). Octyl acetate (0.009, ml, 0.290 mmol) is added, and the reaction mixture is stirred at room temperature. The conversion determined by ¹H-NMR spectroscopy is 12 mol % after a reaction time of 24 hours. *(Balance: KERN ABT 220-5DM; max=220/82 g; d=0.1 mg/0.01 mg) The compounds detected at the end point with reference to the ¹H-NMR spectrum are: 1-octanol, octyl acetate, methyl acetate and methanol having the shift values indicated in Example 13a.

### EXAMPLE 13C

### Reaction from Example 13b at 50° C.

Yttrium(III) tris[hydrogen(pentafluoroethyl)phosphonate], Y[(C₂F₅)₂P(O)₂OH]₃ (7.55 mg*, 0.011 mmol), is dissolved in CD₃OD (0.5 ml). Octyl acetate (0.022 ml, 0.110 mmol) is added, and the reaction mixture is stirred at an oil-bath temperature of 50° C. The conversion determined by ¹H-NMR spectroscopy is 65 mol % after a reaction time of 24 hours. *(Balance: KERN ABT 220-5DM; max=220/82 g; d=0.1 mg/0.01 mg)

The compounds detected at the end point with reference to the ¹H-NMR spectrum are: 1-octanol, octyl acetate, methyl acetate and methanol having the shift values indicated in Example 13a.

### EXAMPLE 14

### Synthesis of 1-(4-ethoxyphenyl)propan-1-one by reaction of ethoxybenzene with propionic anhydride, Catalysed by Al[C₂F₅P(O)₂OH]₃ (5 mol %)

Aluminium(III) tris(pentafluoroethylhydrogenphosphonate) (0.312 g, 0.05 mmol) is dissolved in propionic anhydride (1.65 g, 12.7 mmol) in a 50 ml two-necked flask with reflux condenser. A clear solution is present. Ethoxybenzene (1.222 g, 10 mmol) is added, and the solution is stirred at 70° C. (temperature in the solution) for 2 h. The conversion of ethoxybenzene determined by ¹H-NMR spectroscopy is 74%.

NMR (solvent: CD₃CN; δ in ppm):

¹H NMR: 7.91 (m, 2H, —CH═), 6.94 (m, 2H, —CH═), 4.08 (q, ³J_{H,H}=7.0 Hz, 2H, O—C—CH₂—), 2.94 (q, ³J_{H,H}=7.3 Hz, 2H, O═C—CH₂—), 1.37 (t, ³J_{H,H}=7.0 Hz, 3H, —CH₃), 1.07 (t, ³J_{H,H}=7.6 Hz, 3H, —CH₃).

1. Compounds of the formula I
[Kt]^{z+}z[R_{f}P(O)(OH)O]⁻ I,
where
R_{f} corresponds to the formula CₙF₍₂ₙ₊₁₎₋₍ₘ₊ₖ₎HₘXₖ,
X denotes Cl, Br or I,
n denotes 1, 2, 3, 4, 5, 6, 7, 8, 9, 10, 11 or 12,
m denotes 0, 1, 2, 3 or 4,
k denotes 0, 1 or 2,
with the proviso that, if n denotes 1, m denotes 0, 1 or 2 and/or k denotes 0, 1 or 2 and
[Kt]^{z+} denotes a singly, doubly, triply or multiply positively charged metal atom.

2. Compounds according to claim 1, where R_{f} denotes a linear or branched perfluoroalkyl group having 1 to 12 C atoms.

3. Compounds according to claim 1, where [Kt]^{z+} is selected from cations of the metals Li, Na, Ca, Mg, Ag, Fe, Co, Ni, Cu, Au, Al, In, Sn, Zn, Bi, Rh, Ru, Ir, Pd, Pt, Os, Cr, Mo, W, V, Nb, Ta, Ti, Zr, Hf, Y, Yb, La, Sc, Lu, Ce, Nd, Tb, Er, Eu or Sm.

4. Process for the preparation of compounds of the formula I according to claim 1, characterised in that a compound of the formula II
R_{f}P(O)(OH)₂ II,
where R_{f} has a meaning indicated in claim 1,
is reacted with a metal which forms the future metal cation [Kt]^{z+}, or a compound of the formula III
[Kt]^{z+}z/b[An]^{b-} III,
where [Kt]^{z+} has a meaning indicated in claim 1, and
[An]^{b-} denotes [Cl]⁻, [OH]⁻, [CO₃]²⁻ or [O]²⁻,
where equimolar amounts of compounds of the formula II and of the formula III or of the metal are employed, which is determined by the valence of the cation [Kt]^{z+} in the compound of the formula I.

5. Process according to claim 4, characterised in that the reaction is carried out in the presence or without the presence of water or an organic solvent.

6. Process for the preparation of the compounds of the formula I according to claim 1, characterised in that a compound of the formula IV
[Kt]^{z+}z/2[R_{f}P(O)O₂]²⁻ IV,
is reacted in a disproportionation reaction with a compound of the formula II
R_{f}P(O)(OH)₂ II,
where
R_{f} corresponds to the formula CₙF₍₂ₙ₊₁₎₋₍ₘ₊ₖ₎HₘXₖ,
X denotes Cl, Br or I,
n denotes 1, 2, 3, 4, 5, 6, 7, 8, 9, 10, 11 or 12,
m denotes 0, 1, 2, 3 or 4,
k denotes 0, 1 or 2,
with the proviso that, if n denotes 1, m denotes 0, 1 or 2 and/or k denotes 0, 1 or 2 and
[Kt]^{z+} denotes a singly, doubly, triply or multiply positively charged metal atom.

7. A method for performing catalysis which comprises using compounds of the formula I according to claim 1 as bifunctional catalysts.

8. Bifunctional catalysts of the formula I according to claim 1 for use in organic synthesis.

9. Bifunctional catalysts of the formula I according to claim 8 for use in Lewis acid-catalysed and/or Brønsted acid-catalysed reactions or domino reactions selected from a condensation reaction, alcoholysis, aldol reaction, Mukaiyama aldol reaction, Gattermann-Koch reaction, Beckmann and Fries rearrangement, Friedel-Crafts acylation, Friedel-Crafts alkylation, Mannich reaction, Diels-Alder reaction, aza-Diels-Alder reaction, Baylis-Hillman reaction, Reformatsky reaction, Claisen rearrangement, Prins cyclisation reaction, allylation of carbonyl compounds, cyanation of aldehydes and ketones, cyanosilylation of aldehydes and ketones, 1,3-dipolar cycloaddition, hydration of alkenes, cyclisation reaction, polymerisation, Michael reaction, oxidation and reduction reactions.
